(19) United States
(12) Patent Application Publication
(10) Pub. No.: US 2013/0095196 A1
(43) Pub. Date: Apr. 18, 2013

(54) Blended oil skin moisturizer and skin repairer

(71) Applicant: Sherry May Raymond - Coblantz, Bend, OR (US)

(72) Inventor: Sherry May Raymond - Coblantz, Bend, OR (US)

(21) Appl. No.: 13/317,346

(22) Filed: Oct. 17, 2011

(51) Int. Cl. A61K 8/97; A61Q 19/00

(52) U.S. Cl. 424/729

(57) ABSTRACT

An organic skin moisturizer comprised of Sea Buckthorn seed oil, Camellia seed oil, Argan seed oil, Pomegranate seed oil, Rosemary essential oil, d-alpha tocopherol, Tea Tree oil, Helichrysum oil, vitamin A palmitate. A lighter organic skin moisturizer comprised of Sea Buckthorn seed oil, Camellia seed oil, Argan seed oil, Pomegranate seed oil, Meadowfoam seed oil, Jojoba oil, d-alpha tocopherol, and Rosemary essential oil. And a damaged skin moisturizer, comprised of Sea Buckthorn seed oil, Tamanu oil, Meadowfoam seed oil, d-alpha tocopherol, Tea tree oil, and Vetiver oil.

### CROSS-REFERENCE TO RELATED APPLICATIONS

None.

### STATEMENT REGARDING FEDERALLY SPONSORED RESEARCH OR DEVELOPMENT

None.

### BACKGROUND OF THE INVENTION

The present invention relates to a composition for application on the skin for the treatment of wrinkled, damaged and dry skin. The composition has been found to be especially effective against skin blemishes, rosacea, eczema, wrinkles, dryness and contains properties of stimulation to tissue regeneration and epithelial healing with analgesic and anti-inflammation and anti-fungal effects but without leaving scars. The composition is a most welcome remedy for bum, cuts, frostbites, and sores. This anti-aging formula has been used to produce soft moist skin and can be used on an everyday basis without any negative affects on the skin or body. The composition promotes a firmness to the skin and dermal hydration.

There are many products and formulas for treating dry rough skin and just as many formulas to fight wrinkles. The prior art is replete with products and patents claiming skin protection and salvations for aging skin however the present invention combines a combination of organic ingredients for treating various skin disorders that is unique and effective in bringing about soft, moist skin and reducing wrinkles without harming the skin or the body.

Skin disorders, as the term is used herein, encompasses numerous skin conditions ranging in severity from severe dermatitis, severe dry skin, psoriasis, rosacea and other skin blemishes to less severe conditions, such as lack of adequate skin firmness, dermal hydration or scaling, which are nonetheless unsightly and may cause physical discomfort.

Until now, the treatment of skin disorders has been largely based on non-specific drugs, and only limited success has been achieved. Dermatitis, for example, which may be accompanied by severe scaling, fissures, edema, oozing, erosion, itching and thickening of the skin, commonly has been treated with corticosteroids. Such compounds provide symptomatic relief for some patients. Steroids, however, are known to produce many local and systemic side effects, and their long term use may not be desirable.

Similarly Vitamin D is therapeutically effective in treating certain skin disorders, but only in dosages which are associated with undesirable side effects. Vitamin D at the dose ranges used in currently marketed topical preparations is not therapeutically effective against contact dermatitis. Other formulations for treating skin disorders have either been ineffective or have caused significant irritation to the skin. Several formulations have been proposed to overcome the disadvantages of the prior art, both for treating skin disorders, and for use in cosmetics in order to prevent skin irritation and clear blemishes.

There are numerous patents issued for the care and condition of skin. Each patent uses a variety of ingredients with the combination providing some form of skin treatment. The list is exhaustive, and includes such patents as: U.S. Pat. No. 7,306,810 issued Dec. 11, 2007 to Spencer discloses a skin cream which comprises at least one anti-oxidant, an anti-inflammatory agent, an exfoliant, and an agent to protect against UV irradiation and the cream is made from either oils or creams to promote adsorption of the active ingredients and to increase vibrancy of the skin tone. U.S. Pat. No. 6,281,236 issued Aug. 28, 2001 to Farber discloses a skin cream composition containing allantoin and an emulsifier with improved stability coming from the adjustment of the pH to a range of 4.5 to 5.8. The lower pH preserves the stability of the allantoin and the functionality of the emulsifier system is maintained. U.S. Pat. No. 6,572,868, issued Jun. 3, 2003 to Sandra E. Cope, discloses a restructuring complex for cosmetic compositions. The composition comprises safe and effective amounts of carrageenans, borage seed oil, squalane, ceramide 3, ceramide 6, red algae extract, dipalmitoyl hydroxproline, and oleuropein. U.S. Pat. No. 6,193,987, issued Feb. 27, 2001 to M. H. Harbeck, discloses a lubricating composition for the hands and skin. The composition has as its constituents a mixture of organic safflower oil, flaxseed oil, tincture of benzoin, and organic beeswax. And, U.S. Pat. No. 6,479,043, issued Nov. 12, 2002 to Tietjen et al., discloses a depilatory composition. The composition includes emollients, skin conditioners, buffering agents, viscosity increasing agents, emulsion stabilizers, pH adjusters, chelating agents, fragrance, color, lubricants, propellants, or biological agents.

Various topical formulations and oral regimens of vitamins and herbs have been proposed for the treatment of skin conditions. U.S. Pat. No. 6,228,387, issued May 8, 2001 to M. Borod, describes a first composition for topical application and a second composition for oral administration for the treatment of hemorrhoids. The topical composition includes several herbs and vitamins, including grape seed extract and vitamin E, and in one embodiment, a few drops of Essential Oil of Chamomile. Vitamin E occurs naturally as a mixture of tocopherols, the most active being a-tocopherol, used externally, vitamin E is healing to the skin, being used for protection from sun damage, reducing facial lines and wrinkles, and improving skin smoothness, being used as an additive to massage oils and face creams.

In the prior art, there is little or no distinction between the production of the various compound used in the numerous skin treatments. The present invention provides for a combination of organic ingredients blended from various oils to provide for a unique and exceptional anti-wrinkle formula that can be used everyday with no adverse side effects. The use of organic products, including wild field products eliminates additives such as synthetic pesticides and chemical fertilizers. The products used in the blended compound do not contain genetically modified organisms, and are not processed using irradiation, industrial solvents, or chemical food additives. By eliminating products that are made or produced with pesticides, genetically altered organisms or chemical fertilizer the effects of the product are enhanced and this limits any potential harmful side effects to the skin.

### BRIEF SUMMARY OF THE INVENTION

A night moisturizer, comprising approximately: 30-40% Sea Buckthorn seed oil, 17-25% Camellia seed oil, 18-24% Argan seed oil, 5-12% Pomegranate seed oil, 5-10% Rosemary extract oil, 1-5% d-alpha tocopherol, 0.5-4% Tea Tree oil, 0.5-4% Helichrysum oil, and less than 1% of vitamin A palmitate. All of the ingredients of this skin product are organically or naturally produced and processed.

A day moisturizer, comprising approximately: 20-35% Sea Buckthorn seed oil, 17-25% Camellia seed oil, 15-24% Argan seed oil, 5-15% Pomegranate seed oil, 5-12% Meadowfoam seed oil, 2-6% Jojoba oil, 3-7% d-alpha tocopherol, and 1-5% Rosemary essential oil.

Finally, a damaged skin moisturizer, comprising approximately; 50-65% Sea Buckthorn seed oil, 17-24% Tamanu oil, 10-15%, Meadowfoam seed oil, 3-8% d-alpha tocopherol, 1-4% Tea tree oil, or melaleuca oil, and 0.5-3% Vetiver oil.

The above percentages are volume measurements of the various components added making up the moisturizers. The various oils are blended at room temperature or approximately 60° to 80° Fahrenheit with low humidity. The oils are first filtered prior to blending. Application of the moisturizers should be done after the skin has been washed with a hot washcloth. The blended oils are applied to the fingertips or palm and applied in an upward motion or against the grain of the skin. If makeup or sun screen are to be applied after application of the moisturizer, then wait approximately 20 to 30 minutes or until the moisturizer has been absorbed by the skin.

### BRIEF DESCRIPTION OF THE SEVERAL VIEWS OF THE DRAWING

None

### DETAILED DESCRIPTION OF THE INVENTION

Various embodiments of the invention will now be described. The following descriptions provide specific details for a thorough understanding and enabling description of these embodiments. Additionally, some well-known structures or functions may not be shown or described in detail, so as to avoid unnecessarily obscuring the relevant description of the various aspects and embodiments of the invention.

The terminology used in the description presented below is intended to be interpreted in its broadest reasonable manner, even though it is being used in conjunction with a detailed description of certain specific embodiments of the invention. Certain terms may even be emphasized herein; however, any terminology intended to be interpreted in any restricted manner will be overly and specifically defined as such in this Detailed Description section.

The present invention is a topical blended oil created by combining several organically or naturally produced ingredients, most all of these ingredients are plant based. The various ingredients are oils that are blended at room temperature or warmed after being filtered to remove any non-essential impurities. By combining the various ingredients the applicant has found significant improvement in a user's skin. The skin is more youthful, firmer, softer and shows fewer wrinkles. The present invention is a topical skin treatment that reduces dryness and removes or reduces wrinkles. The present invention is beneficial to sunburned skin or skin with blemishes due to harsh environments, such as sun, wind, dryness and other skin conditions. The present invention produces a firmer tighter softer skin with fewer wrinkles and more pleasing appearance.

The present invention is a series of skin moisturizers made of blended oils. The present invention is not an emulsion and is not a water based moisturizer. Each blended oil moisturizer discussed below uses primarily carrier and essential oils. The blending of the two types of oils allows for the oils to be more readily blended and more readily absorbed into the skin. The ingredients in the moisturizers described below are made mostly from organic products and organic processing.

The moisturizers are produced using organic products, which means: the ingredients are grown and processed organically. For example, organic farming practices are designed to encourage soil and water conservation and reduce pollution. Farmers who grow organic produce don't use conventional methods to fertilize or control weeds. For example, rather than using chemical weed killers, organic farmers may conduct more sophisticated crop rotations and spread mulch or manure to keep weeds at bay. Additionally, the ingredients are organically processed which includes; cold press extraction or other means for processing the various plants to produces the oils without using or adding chemicals in the extraction process. The following moisturizers were developed using organic or natural ingredients and have produced exceptional results. The claims are not limited to organic ingredients but the applicant cannot confirm the same positive results using non-organic products.

A night moisturizer, comprising approximately: 30-40% SeaBuckthorn seed oil, 17-25% Camellia seed oil, 18-24% Argan seed oil, 5-12% Pomegranate seed oil, 5-10% Rosemary extract oil, 1-5% d-alpha tocopherol, 0.5-4% Tea Tree oil, 0.5-4% Helichrysum oil, and less than 1% of vitamin A palmitate. All of the ingredients of this skin product are organically or naturally produced and processed and come from many countries.

The base of the composition of the night moisturizer is the 1-5% d-alpha tocopherol. The amount of d-alpha tocopherol, as well as the other listed ingredients, is by volume. Since the specific weights and densities of the various oils are similar volume measurements closely represent percentages by weight. The various oils, both essential and carrier oils are blended at room temperature or warmed, approximately 60° to 90° Fahrenheit in a low humidity environment. The d-alpha tocopherol, the Rosemary extract, and Tea Tree oil contributes to the stability of the blended oils and are high in antioxidants. The tocopherol is known to protect the skin from UV rays and reactive radicals. The tocopherol also provides some moisturizing benefits to the skin.

30-40% Sea Buckthorn seed oil which is derived from the seed of the Hippophae rhamnoides, a plant or shrub found throughout Europe and Asia. The oil is produced by cold press of the seeds and only the seeds which produces a number of fatty acids, including; Beta Carotene, Lycopene, Linoleic, Oleic, Palmitic, Palmitoleic, and Stearic acids. In addition to being used as a conditioning oil it is also beneficial in the treatment of burns, wounds, lesions, and sun damaged skin.

17-25% Camellia seed oil is a highly penetrating and virtually odorless oil that possesses a slight herbal aroma. It is virtually clear with a slight yellow hue. Camellia Oil is light in texture and absorbs quickly into the skin. Approximately 85% of the fatty acid composition contained in Camellia Oil is in the form of monounsaturated oleic acid. Camellia Oil is also a rich source of other omega 3, 6 and 9 fatty acids plus Vitamin E and polyphenols. The Camellia seed oil has been shown to have the following properties; penetrates quickly, moisturizes, emits a light herbal aroma, is rich in fatty and essential fatty acids, contains tocopherol a form of Vitamin E, and contains an abundance of polyphenols.

18-24% Argan oil is an oil produced from the kernels of the Argan tree, found primarily in Morocco. The oil is valued for its nutritive, cosmetic and numerous medicinal properties. Argan oil remains one of the rarest oils in the world due to the small and very specific growing areas. The oil contains a number of fatty acids, including; Palmitic, Stearic, Oleic, Linoleic, and Linolenic acid. The oil is exceptionally rich in natural tocopherols, phenols and phenolic acid, carotenes, squalene, essential fatty acids, 80% unsaturated fatty acids. The composition of the Argan oil makes it resistant to oxidation and therefore a stabilizer for the blended oils.

5-12% Pomegranate seed oil which is commonly used in cosmetic products to revitalize dull or mature skin, assist with wrinkles, and to soothe minor skin irritations. It provides relief from minor skin irritations and inflammation, including dry skin, eczema, psoriasis and sunburned skin. The conjugated fatty acids give it strong anti-inflammatory properties, which help to reduce swelling and ease muscular aches and pains. Several recent studies have shown that pomegranate seed oil stimulates keratinocyte proliferation, promoting regeneration and strengthening of the epidermis. The oil contains more than 60% puninic acid, a valuable conjugated linolenic acid. Additional ingredients of the Pomegranate seed oil, include tocopherols, sterols and a small amount of squalene.

5-10% Rosemary essential oil improves circulation and has a rejuvenating effect on the skin. Rosemary is a common ingredient used in many cosmetics, including skin toners, creams, soaps and hair products. The essential oil is known to assist in the prevention of age-related skin damage, such as wrinkles. The oil is derived from the flowering top of the plant. Organic extraction requires steam distillation of the oil from the plant.

0.5-3% Helichrysum italicum oil which has properties that are beneficial to the skin, including; acting as an anti-inflammatory and soothes burns and raw chapped skin. It is used as a stabilizing and preserving agent in the present compound as well as a contributing to the benefits to the skin.

And, less than 1% of vitamin A palmitate is associated with development of mature epidermal cells creating or promoting newer fresher skin.

The above blended oils are then applied topically to the skin. The blended oils can be applied every day or as needed. The skin should be washed with a hot wash cloth and the blended oil placed on the fingertips or palm and applied to the skin against the grain of the skin in an upward motion. The compound should be allowed to time to be absorbed by the skin and will depend upon the skin type but approximately 20 minutes or less. After absorption, other skin products, such as makeup or sun screen, can be applied. The night moisturizer is best applied at night prior to going to bed.

A preferred composition for the night moisturize, includes approximately: 32% Sea Buckthorn seed oil, 20% Camellia seed oil, 20% Argan seed oil, 10% Pomegranate seed oil, 7% Rosemary extract oil, 4.4% d-alpha tocopherol, 3% Tea Tree oil, 3% Helichrysum oil, and less than 1% of vitamin A palmitate.

Most of the blended oils of the present invention are either designated essential oils or carrier oils. Essential oils are extracts from the root, bark, stem, leaves and aromatic portions of the plant. There are several extraction methods in practice like the steam distillation method, cold-pressing method and solvent extraction method. These essential oils are thin oils with strong aromas. They evaporate quickly and generally absorb quickly into the skin. Carrier oils, also called base oils, are vegetable oils that are used to dilute essential oils. Since essential oils can cause skin irritations or itching when used in undiluted forms. Carrier oils are used as base oils to dilute essential oils. In the present compounds, the following oils are carrier oils; pomegranate seed oil, argan oil, sea buckthorn seed oil, camellia seed oil, meadowfoam seed oil, and d-alpha tocopherol. The essential oils in the present invention, include; helichrysum italicum oil, and tea tree oil. Vetiver oil used in the compound described below is an essential oil.

A day moisturizer comprising most of the ingredients in the night moisturizer can be made by comprising approximately: 20-35% Sea Buckthorn seed oil, 17-25% Camellia seed oil, 15-24% Argan seed oil, 5-15% Pomegranate seed oil, 5-12% Meadowfoam seed oil, 2-6% Jojoba oil, 3-7% d-alpha tocopherol, and 1-5% Rosemary essential oil.

The blended oils provide a lighter moisturizer and is more readily absorbed into the skin. The same method for applying the night moisturizer is repeated for the day moisturizer. The day moisturizer has two additional oil blended to the base, including; meadowfoam seed oil and jojoba oil. Three of the blended oils are not present in the day moisturizer, including; tea tree oil, helichrysum oil and vitamin A palmitate.

Meadowfoam seed oil is extracted from the seeds of Limnanthes alba (meadowfoam). The seeds contain 20-30% oil. Meadowfoam seed oil is extraordinarily stable, primarily because it contains over 98% long chain fatty acids. Meadowfoam oil is used in cosmetic and hair-care applications due to its stability, lubricity and ability to stay on the skin.

Jojoba oil is a mixture of long chain monounsaturated liquid wax esters. Jojoba oil is extracted from seeds of the jojoba plant, scientifically known as Simmondsia chinensis, which are indigenous to southern United States and northern Mexico. Jojoba oil has a golden color whereas processed jojoba oil is clear and transparent. It has a relatively long shelf life as it is a relatively stable liquid. It does not oxidize easily and will not turn rancid compared to other oils.

A preferred composition for the daily moisturizer, comprises approximately: 26% Sea Buckthorn seed oil, 24% Camellia seed oil, 18% Argan seed oil, 12% Pomegranate seed oil, 8% Meadowfoam seed oil, 4% Jojoba oil, 5% d-alpha tocopherol, and 3% Rosemary extract oil.

A damaged skin moisturizer uses several of the ingredients of the night moisturizer along with Tamanu and Vetiver oils. The damaged skin moisturizer is comprised of approximately: 50-65% Sea Buckthorn seed oil, 17-24% Tamanu oil, 10-15%, Meadowfoam seed oil, 3-8% d-alpha tocopherol, 1-4% Tea tree oil, or melaleuca oil, and 0.5-3% Vetiver oil.

The damaged skin moisturizer is blended like the night moisturizer without pomegranate seed oil, camellia seed oil, rosemary essential oil, argan oil, and helichrysum oil. Additional oils are blended in, including; tamanu oil, meadowfoam seed oil and vetiver oil.

The tamanu oil comes from the Calophyllum inophyllum which is a large evergreen tree. In Tahiti the tree is called the Tamanu tree and the desired oil is extracted from the kernel of the nut. The oil is a rich, thick green oil, however it is easily and completely absorbed into the skin. Once applied, it leaves the skin feeling smooth, plump and soft, with no greasiness, it also adds a healthy glow to the skin. Calophyllum has a mild and pleasant aroma and can be easily mixed with most essential oils.

The Vetiver oil is an essential oil distilled from the roots of the Chrysopogon zizanioides. The plant is native to India but is cultivated throughout the tropical zones around the world. The oil has excellent fixative properties and is used widely in perfumes or other scented skin compounds. Vetiver oil is an Essential oil containing over 100 identified components. The oil is amber brown and rather thick. The odor of vetiver oil is described as deep, sweet, woody, smoky, earthy, amber, balsam and very strong.

The damaged skin moisturizer has a strong aromatic smell and is applied topically and allowed to sit on the skin for at least 24 hours.

A preferred skin repair formula, comprises approximately; 60% Sea Buckthorn seed oil, 20% Tamanu oil, 11%, Meadowfoam seed oil, 5 d-alpha tocopherol, 3% Tea tree oil, or melaleuca oil, and 1% Vetiver oil.

Another form of the moisturizer can be made by into a hand and body lotion by comprising: 25-35% Sunflower seed oil; 20-26% Aloe Vera gel; 7-13% Sea Buckthorn seed oil; 7-10% vegetable Glycerin; 4-8% Camellia seed oil; 3-7% emulsifying wax; 3-7% Rosemary essential oil; 1-6% d-alpha tocopherol; 1-6% Jojoba oil; 1-4% Stearic acid.

Sunflower oil is mainly a triglyceride, a typical constituent is shown. Palmitic acid: 4-9%; Stearic acid: 1-7%; Oleic acid: 14-10%; Linoleic acid: 48 -74%. Sunflower oil is mainly triglycerides (fats), typically derived from the fatty acids linoleic acid (with is doubly unsaturated) and oleic acid. The sunflower oil can retain moisture in the skin. It may also provide a protective barrier that resists infection in premature infants. Studies using sunflower oil have been conducted involving low birth weight infants who are often susceptible to infection due to their underdeveloped skin.

The Aloe vera gel is a product made from the pulp of the meaty leaves of Aloe vera and has been used topically for thousands of years to treat wounds, skin infections, burns, and numerous other dermatologic conditions. Dried latex from the inner lining of the leaf has traditionally been used as an oral laxative.

A preferred embodiment of the hand and body lotion is comprised of approximately: 30% Sunflower seed oil; 24% Aloe Vera gel; 10% Sea Buckthorn seed oil; 10% vegetable Glycerin; 6% Camellia seed oil; 4% emulsifying wax; 5% Rosemary essential oil; 4% d-alpha tocopherol; 4% Jojoba oil; 3% Stearic acid.

I claim:

1. A night moisturizer comprising:
30-40% by volume Sea Buckthorn seed oil;
17-25% by volume Camellia seed oil;
18-24% Argan seed oil;
5-12% by volume Pomegranate seed oil;
5-10% by volume Rosemary extract oil;
1-5% by volume d-alpha tocopherol;
0.5-4% by volume Tea Tree oil;
0.5-4% by volume Helichrysum oil; and
less than 1% by volume of vitamin A palmitate.

2. The night moisturizer of claim 1, wherein the Sea Buckthorn seed oil is 32%; the Camellia seed oil is 20%; the Argan seed oil is 20%, the Pomegranate seed oil is 10%; the Rosemary essential oil is 7%; the d-alpha tocopherol is 4.4%; the Tea Tree oil is 3%; the Helichrysum oil is 3%; and the vitamin A palmitate is 0.6%.

3. A day moisturizer comprising:
20-35% by volume Sea Buckthorn seed oil;
17-25% by volume Camellia seed oil;
15-24% by volume Argan seed oil;
5-15% by volume Pomegranate seed oil;
5-12% by volume Meadowfoam seed oil;
2-6% by volume Jojoba oil;
3-7% by volume d-alpha tocopherol; and
1-5% by volume Rosemary essential oil.

4. The day moisturizer of claim 3 wherein the Sea Buckthorn seed oil is 26%; the Camellia seed oil is 24%; the Argan seed oil is 18%; the Pomegranate seed oil is 12%; the Meadowfoam seed oil is 8%; the Jojoba oil is 4%; the d-alpha tocopherol is 5%; and the Rosemary essential oil is 3%.

5. A damaged skin moisturizer comprising:
50-65% by volume Sea Buckthorn seed oil;
17-24% by volume Tamanu oil;
10-15%, by volume Meadowfoam seed oil;
3-8% by volume d-alpha tocopherol;
1-4% by volume Tea tree oil; and
0.5-3% by volume Vetiver oil.

6. The damaged skin moisturizer of claim 5 wherein the Sea Buckthorn seed oil is 60%; the Tamanu oil is 20%; the Meadowfoam seed oil is 11%; the d-alpha tocopherol is approximately 5%; the Tea tree oil is 3%; and the Vetiver oil is 1%.

7. A hand and body lotion comprising:
25-35% Sunflower seed oil;
20-26% Aloe Vera gel;
7-13% Sea Buckthorn seed oil;
7-10% vegetable Glycerin;
4-8% Camellia seed oil;
3-7% emulsifying wax;
3-7% Rosemary essential oil;
1-6% d-alpha tocopherol;
1-6% Jojoba oil; and
1-4% Stearic acid.

8. The hand and body lotion of claim 7, wherein the Sunflower seed oil is 30%; the Aloe Vera gel is 24%; the Sea Buckthorn seed oil is 10%; the vegetable Glycerin is 10%; the Camellia seed oil is 6%; the emulsifying wax is 4%; the Rosemary essential oil is 5%; the d-alpha tocopherol 4%; the Jojoba oil is 4%; the Stearic acid is 3%.
